(19) United States
(12) Patent Application Publication
(10) Pub. No.: US 2014/0148631 A1
(43) Pub. Date: May 29, 2014

(54) PROCESS FOR PREPARING ETHYLENE AND/OR PROPYLENE

(71) Applicant: SHELL OIL COMPANY, Houston, TX (US)

(72) Inventors: Leslie Andrew CHEWTER, Amsterdam (NL); Ye-Mon CHEN, Sugar Land, TX (US); Timothy NISBET, Amster (NL); Sivakumar SADASIVAN VIJAYAKUMARI, Amsterdam (NL); Jeroen VAN WESTRENEN, Amsterdam (NL)

(21) Appl. No.: 13/932,296

(22) Filed: Jul. 1, 2013

Related U.S. Application Data
(60) Provisional application No. 61/667,641, filed on Jul. 3, 2012.

(51) Int. Cl. C07C 1/22

(52) U.S. Cl. CPC C07C 1/22
USPC 585/640; 422/607

(57) ABSTRACT

The present invention provides a process for preparing ethylene and/or propylene, wherein an oxygenate feedstock is contacted with a zeolite-comprising catalyst at a temperature in the range of from 500 to 700° C. to obtain a reactor effluent comprising ethylene and/or propylene and the oxygenate feedstock is contacted with the catalyst in a riser reactor having a reactor wall defining a flow trajectory towards a downstream outlet for reactor effluent, wherein at least oxygenate feedstock and catalyst are provided at one or more upstream inlets of the riser reactor and wherein C5 olefins are admitted to the riser reactor at one or more of locations along the length of the flow trajectory. The invention further provides a reaction system suitable for preparing ethylene and propylene.

This application claims the benefit of U.S. Application No. 61/667,641 filed Jul. 3, 2012 which is incorporated herein by reference.

### FIELD OF THE INVENTION

This invention relates to a process for preparing ethylene and/or propylene and a reaction system suitable therefore.

### BACKGROUND TO THE INVENTION

Conventionally, ethylene and propylene are produced via steam cracking of paraffinic feedstocks including ethane, propane, naphtha and hydrowax. An alternative route to ethylene and propylene is an oxygenate-to-olefin (OTO) process. Interest in OTO processes for producing ethylene and propylene is growing in view of the increasing availability of natural gas. Methane in the natural gas can be converted, for instance, to methanol or dimethylether (DME), both of which are suitable feedstocks for an OTO process.

In an OTO process, an oxygenate such as methanol is provided to a reaction zone comprising a suitable conversion catalyst and converted to ethylene and propylene.

The conversion of oxygenates, such as methanol, to olefins is an exothermic process. Consequently, as the conversion process progresses, the temperature of the reaction mixture in the reactor increases. Such a temperature increase is undesired as it may accelerate deactivation of the catalyst.

U.S. Pat. No. 4,071,573 describes the deactivation of zeolite-comprising catalysts due to the temperature increase in exothermic methanol to gasoline processes. In the process of U.S. Pat. No. 4,071,573, the temperature in the conversion reactor is controlled in several ways including addition of a quench medium to the reaction mixture inside the reactor or cooling of the catalyst prior to entering the reactor.

In US2009/0163756, an alternative method for removing heat from an oxygenate conversion process is described. In the process of US2009/0163756, cooling tubes are disposed within the reactor, extending adjacent to the reactor wall from an upper part of the reactor to a lower part of the reactor. A cooling medium is passed through the cooling tubes to remove the heat of reaction.

A disadvantage of the prior art processes is that, although the temperature inside the reactor may be reduced, valuable heat is lost as it is withdrawn from the process.

There is a need in the art for a process for producing ethylene and/or propylene from an oxygenate feedstock, wherein the temperature of the reaction mixture, and in particular the catalyst, in the reactor is controlled during an exothermic oxygenate to olefins process. While at the same time, using the heat of reaction generated by the exothermic oxygenate to olefins process to produce further ethylene and propylene.

### SUMMARY OF THE INVENTION

It has now been found that in an oxygenate to olefins process, the temperature of the reaction mixture, and in particular the catalyst, in the reactor may be controlled, by providing C5 olefins to the process when at least part of the oxygenates have already been converted. The C5 olefins are endothermically cracked to ethylene and propylene using the heat of reaction generated by the exothermic oxygenate to olefins conversion.

Accordingly, the present invention provides a process for preparing ethylene and/or propylene, wherein an oxygenate feedstock is contacted with a zeolite-comprising catalyst at a temperature in the range of from 500 to 700° C. to obtain a reactor effluent comprising ethylene and/or propylene and the oxygenate feedstock is contacted with the zeolite-comprising catalyst in a riser reactor having a reactor wall defining a flow trajectory towards a downstream outlet for reactor effluent, wherein at least oxygenate feedstock and catalyst are provided at one or more upstream inlets of the riser reactor and wherein C5 olefins are admitted to the riser reactor at one or more locations along the length of the flow trajectory.

Reference herein to an oxygenate feedstock is to a feedstock comprising oxygenates.

Reference herein to C5 olefins is to olefins having 5 carbon atoms.

The conversion of the oxygenate feedstock over a zeolite-comprising catalyst to at least ethylene and/or propylene is also referred to as an oxygenate to olefin (OTO) process. Such OTO processes are well known in the art.

The process according to the invention allows for the control of the temperature of the reaction mixture passing through the reactor, and in particular the temperature of the catalyst in the reactor during an oxygenate to olefins process. Where, in prior art processes, heat generated by the exothermic conversion of oxygenates to olefins is removed from the process by for instance a quench medium or cooling medium, in the process according to the present invention this heat is used to crack C5 olefins. These C5 olefins are provided separately to the process. By using the heat generated by the exothermic conversion of oxygenates to olefins to crack C5 olefins, additional ethylene and propylene may be produced.

One advantage of the process according to the present invention is that catalyst deactivation due to the exposure of the catalyst to high temperatures may be reduced by controlling the temperature of the reaction mixture in the reactor and maintaining the temperature within an acceptable temperature range. An additional advantage of the process according to the present invention is that operating the process within a narrow temperature range may be beneficial to the selectivity of the process, i.e. result in less coke make and reduced formation of paraffins, compared to a process that is operated over a wide temperature range of the reaction mixture. A narrow temperature range increases the predictability of the process and reduces the risk of hotspots inside the reactor. In another aspect, the invention provides a reaction system suitable for preparing ethylene and propylene, comprising a riser reactor comprising:

a) an inlet for oxygenate feedstock;
b) an inlet for zeolite-comprising catalyst;
d) a reactor wall defining a flow trajectory from the inlets for oxygenate feedstock and zeolite catalyst to the outlet for reactor effluent; and
e) at least one inlet array for providing C5 olefins into the reactor, integrated with the reactor wall.

### BRIEF DESCRIPTION OF THE DRAWING

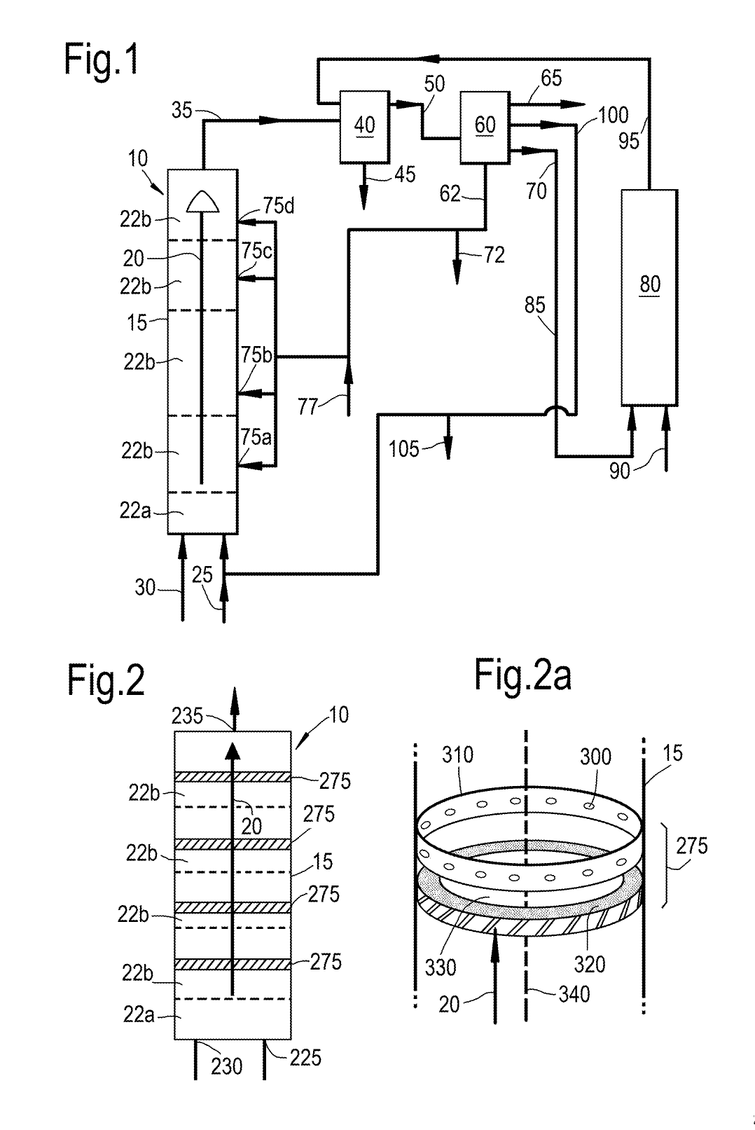

In FIG. 1, a schematic representation is provided of a process according to the invention.

In FIG. 2, an embodiment of a reaction system for preparing ethylene and/or propylene according to the invention is shown.

In FIG. 2a, a preferred embodiment of an inlet array according to the invention is illustrated.

### DETAILED DESCRIPTION OF THE INVENTION

Ethylene and/or propylene can be produced from oxygenates such as methanol and dimethylether (DME) through an oxygenate-to-olefins (OTO) process. Such processes are well known in the art and are also referred to as methanol-to-olefins or methanol-to-propylene processes. In an OTO process, typically the oxygenate is contacted with a zeolite-comprising catalyst at elevated temperatures. In contact with the zeolite-comprising catalyst, the oxygenate is converted into at least ethylene and/or propylene. The conversion of the oxygenate into ethylene and propylene is an exothermic process and resultantly a substantial amount of heat of reaction is released during the conversion of the oxygenate. Unless this heat of reaction is withdrawn from the process, it will cause the temperature of the reaction mixture to increase. This temperature increase may have an undesired effect on the catalyst activity. It is known in the art that increased temperatures induce catalyst deactivation, and therefore it is desired to maintain the temperature increase as small as possible. This may be achieved by utilising the heat of reaction released from the oxygenate conversion to induce an endothermic, i.e. heat consuming, reaction that produces additional ethylene and propylene.

In the process according to the present invention, oxygenates are converted to at least ethylene and propylene by providing an oxygenate feedstock and zeolite-comprising catalyst to a riser reactor and contacting, at elevated temperatures, the oxygenate feedstock and zeolite-comprising catalyst in an initial reaction mixture. The initial reaction mixture comprises oxygenate feedstock and zeolite-comprising catalyst. As the reaction mixture passes through the riser reactor, oxygenate feedstock is consumed while reaction products are formed. Herein both the initial reaction mixture and the mixture formed during the process are referred to as the reaction mixture. As the reaction mixture passes through the riser reactor, C5 olefins are provided to the reaction mixture. In contact with the zeolite-comprising catalyst these C5 olefins are endothermically cracked into ethylene and propylene, while consuming the exothermic heat of reaction released by the oxygenate conversion.

Due to different enthalpic properties of the oxygenate conversion reaction and the C5 cracking reaction, it is possible to control the temperature in the riser reactor during the process.

In the process according to the present invention, ethylene and/or propylene are prepared by contacting an oxygenate feedstock with a zeolite-comprising catalyst at a temperature in the range of from 500 to 700° C. to obtain a reactor effluent comprising ethylene and/or propylene. The reactor effluent will also comprise the zeolite-comprising catalyst and may comprise other hydrocarbons, including oxygenates and C4+ olefins.

In the present invention, the oxygenate feedstock is contacted with the catalyst in a riser reactor. The riser reactor has a reactor wall, which defines a flow trajectory towards a downstream outlet for reactor effluent. Reference herein to the flow trajectory is to a trajectory that the reaction mixture will follow when passed through the riser reactor under normal operation conditions. The riser reactor comprises one or more upstream inlets.

Reference herein to upstream and downstream is to locations along the flow trajectory whereby the flow passes from an upstream location in the direction of a downstream location.

In the process according to the invention, at least oxygenate feedstock and zeolite-comprising catalyst are provided at the one or more upstream inlets of the riser reactor and form the reaction mixture. The reaction mixture may comprise other compounds such as olefins or diluents, which may be provided at the one or more upstream inlets of the riser reactor.

In the process according to the invention, C5 olefins are admitted to the riser reactor at one or more of locations along the length of the flow trajectory. The C5 olefins are admitted to the riser reactor and become part of the reaction mixture existing at the location along the length of the flow trajectory where the C5 olefins are admitted.

These locations where C5 olefins are admitted to the riser reactor are located downstream from the one or more upstream inlets of the riser reactor and upstream of the downstream outlet for reactor effluent. These locations are separate from the one or more upstream inlets of the riser reactor. For the purposes of this specification, the riser reactor may be defined as a riser reactor comprising two or more serially arranged riser reactor stages, such that the reaction mixture passes from one reactor stage downstream into a subsequent riser reactor stage. The first reactor stage herein comprises the one or more upstream inlets for providing oxygenate feedstock and zeolite-comprising catalyst which form the initial reaction mixture, whereas the locations where C5 olefins are admitted to the riser reactor are located downstream of the first riser reactor stage in one or more subsequent riser reactor stages. Therefore, reference herein to C5 olefins that are admitted to the riser reactor at one or more of locations along the length of the flow trajectory is to C5 olefins admitted to reactor at one or more of locations located downstream of the first riser reactor stage in the one or more subsequent riser reactor stages and not C5 olefins that were provided as part of an initial reaction mixture.

It is a particular advantage of the process according to the invention that C5 olefins are admitted to the reactor. C5 olefin cracking is ideally a relatively straightforward process whereby the C5 olefin cracks in contact with the zeolite comprising catalyst into a C2 and a C3 olefin, in particular above 500° C. This cracking reaction can be run at high conversions, up to 100%, while maintaining high ethylene and propylene yields with a low by-product and coke make.

Where, as mentioned above, the cracking of C5 olefins is relatively straightforward and selective for ethylene and propylene, the cracking of other olefins to ethylene and propylene may be less straightforward. Without wishing to be bound by any particular theory, it is believed that the cracking behaviour of C4 olefins and C5 olefins, when contacted with a zeolite-comprising catalyst, is different, in particular above 500° C. The cracking of C4 olefins is an indirect process which involves a primary oligomerisation process to a C8, C12 or higher olefin followed by cracking of the oligomers to lower molecular weight hydrocarbons including ethylene and propylene, but also, amongst other things, to C5 to C7 olefins, and by-products such as C2 to C6 paraffins, cyclic and aromatics. In addition, the cracking of C4 olefins is prone to coke formation, which places a restriction on the desired conversion of the C4 olefins. Generally, paraffins, cyclics and aromatics are not formed by cracking. They are formed by hydrogen transfer reactions, optionally, followed by cyclisation. This is more likely in larger molecules. Hence the C4 olefin cracking process, which, as mentioned above, includes intermediate oligomerisation, is more prone to by-product formation than direct cracking of C5 olefins.

Higher olefins, i.e. C7+ olefins, may be cracked, but are sensitive to the formation of by-products such as C2 to C6 paraffins, cyclics and aromatics. In addition, the cracking of C7+ olefins is prone to coke formation.

Not withstanding the above, C4 olefins may be converted in the process according to the present invention. Without wishing to be bound by any particular theory, it is believed that in the presence of oxygenates, in particular methanol or DME, C4 olefins are alkylated to C5 olefins. These C5 olefins are subsequently converted as described above into at least ethylene and/or propylene. Therefore, it is preferred to add C4 olefins, if any, to the reactor together with or as part of the oxygenate feedstock. When C4 olefins are provided together with the oxygenate feedstock, a maximum amount of oxygenate is available for the preferred reaction of oxygenates with the C4 olefins and the less favoured cracking reaction of C4 olefins is minimised. Where, C4 olefins are provided to the process it is preferred that the oxygenate feedstock comprises methanol or DME.

C6 olefins may follow a cracking mechanism similarly to that of the C5 olefins. Although a C6 olefin may be desirably cracked into two propylene molecules, it may also be cracked into an ethylene and a C4 olefin. As such, the cracking of C6 olefins may produce more C4 olefins compared to the cracking of C5 olefins, however as mentioned above such C4 olefins may be recycled to the process by providing such C4 olefins together with the oxygenate feedstock. As the C6 olefin may desirably crack into two propylene molecules, rather than an ethylene and a propylene molecule as the C5 olefins desirably would, the ethylene to propylene ratio in the reactor effluent may to an extent be tailored by admitting C6 olefins in addition to C5 olefins at the two or more locations along the flow trajectory. Therefore, although C5 olefins are preferred, the process according to the invention may comprise a process wherein at least oxygenate feedstock and catalyst are provided at one or more upstream inlets of the riser reactor and wherein C5 and C6 olefins are admitted to the riser reactor at one or more locations along the length of the flow trajectory.

Preferably, the C5 olefins are admitted at two or more locations along the flow trajectory. More preferably, the C5 olefins are admitted to the riser reactor at in the range of from 2 to 15 locations, even more preferably of from 4 to 10 locations.

As mentioned above, the C5 olefins may crack into ethylene and propylene, however in the presence of oxygenates, in particular methanol or DME, the C5 olefins may also react with the oxygenate and be alkylated to C6 and even higher olefins. As mentioned above, it is preferred to crack C5 olefins rather than C6 olefins. Moreover, it is undesirable to form even higher olefins such as C7 or C8 olefins. Such olefins, as mentioned above, are more prone to coke production and the formation of undesired by-products. Furthermore, when C4 olefins are provided to the process together with the oxygenate feedstock, a reaction of C5 olefins with the oxygenate would compete with the desired reaction between the C4 olefins with the oxygenate. When the availability of oxygenate for the reaction with C4 decreases, C4 olefins present in the reaction mixture may oligomerise to C8 olefins, which as mentioned above is undesirable. Therefore, the C5 olefins are preferably admitted to the reactor at a stage where at least part of the oxygenates in the oxygenates in the oxygenate feedstock have already been converted. Preferably, the C5 olefins are admitted at a stage where at least 50 wt % of the oxygenates have been converted to hydrocarbons other than oxygenates, based on the oxygenates in the feedstock provided to the riser reactor, more preferably at a stage where at least 70 wt % of the oxygenates have been converted. By admitting C5 olefins at a stage where the reaction mixture is depleted in oxygenate, in particular methanol or DME, the C5 olefins may be cracked to obtain the desired reduction of the temperature of the reaction mixture and consequently lower the temperature of the reactor effluent, while producing additional ethylene and propylene rather than undesired by-products.

In order to ensure that the C5 olefins admitted to the riser reactor are well distributed in the reaction mixture, it is preferred to provide a temporary and localized distortion of the flow through the reactor, in particular upstream to the location where the C5 olefins are admitted. The process according to present invention is operated in a riser reactor. The advantage of the use of a riser reactor is that it allows for very accurate control of the contact time of the feed with the catalyst, as riser reactors exhibit a flow of catalyst and reactants through the reactor that approaches plug flow. This type of flow is characterised by a narrow residence time distribution of the catalyst passing through the reactor. Ideally, in a plug flow regime all catalyst particles have the same residence time. However, in reality, friction with the wall of the reactor will result in an accumulation of catalyst at the wall of the reactor, resulting in a broader residence time distribution. The accumulated catalyst, in case of a riser reactor, may flow in an upstream direction along the wall of the reactor, generally referred to as the slip or catalyst slip. In the process according to the invention, it is therefore preferred that the riser reactor comprises one or more obstructing members extending from the inner side of the reactor wall into the flow trajectory. Reference herein to an obstructing member is an object or device that during the process will at least temporarily and in a localized manner distort the flow of the reaction mixture. The obstructing member is preferably a ring shaped device, which when placed inside a riser reactor results in a localised decreased inner diameter of the reactor cross-section. The decreased inner diameter being determined by a circular or oval opening in the obstructing member, wherein the central axis of the opening is aligned with the central axis of the riser reactor. Preferably, the obstructing member is a metal or ceramic ring that is attached to the inner side of the reactor wall. Such a ring structure is preferred over prior art obstructing members such as for instance the grid member of U.S. Pat. No. 4,071,573. These grid members are sensitive to blockage and wear by the zeolite comprising catalyst. Where the flow of slip encounters the obstruction member according the invention, the direction of the slip is diverted toward the centre of the reactor and into the reaction mixture. The catalyst slip is mixed into the reaction mixture. By diverting the catalyst slip back into the reaction mixture, the obstructing members provide means to narrow the residence time distribution of the catalyst in the riser reactor. It has now been found that this mechanism may be utilised to improve the distribution of the C5 olefins admitted to the riser reactor at one or more locations along the flow trajectory. By admitting the C5 olefins to the riser reactor downstream of the obstructing members, the diversion of the catalyst slip along the reactor wall into the reaction mixture will improve the distribution of the C5 olefins within the reaction mixture and thereby favour a homogenous reduction of the temperature of the reaction mixture. The prior art obstructing members such as for instance the grid member of U.S. Pat. No. 4,071,573 will not, or at least to a much lesser extent, divert the slip flow, and consequently may not enhance the distribution of the C5 olefins. The grid like structure affects the flow through the cross-section of the reactor uniformly; whereas the obstructing members according to the present invention only divert the flow near the reactor wall. Preferably, the obstructing member causes the inner diameter to be locally decreased by in the range of from 1 to 25%, preferably of from 2 to 10%, based on the inner diameter of the riser reactor.

To further increase the distribution of the C5 olefins in the reaction mixture, it is preferred that at each location along the length of the flow trajectory where the C5 olefins are admitted to the reactor, the C5 olefins are admitted through a plurality of inlets distributed along the cross-sectional periphery of the reactor wall. Preferably, the provision of C5 olefins is evenly distributed over the plurality of inlets.

The inlets may be integrated in the reactor wall or may be part of an inlet array for providing C5 olefins into the reactor, integrated with the reactor wall and comprising a plurality of inlets for C5 olefins distributed along the periphery of the reactor wall. Reference herein to inlet array integrated with the reactor wall is to an inlet array that is an integral part of the reactor wall or to an inlet array that is arranged on the inner side of the reactor wall along the periphery of the reactor wall.

It is a particular advantage of the process according to the invention that no heat is withdrawn externally from the process to control the temperature inside the reactor, but rather the temperature is controlled by providing the means for endothermic cracking reactions to take place inside the reaction mixture. Preferably, the process is operated under adiabatic conditions. Preferably, the zeolite-comprising catalyst is provided to the riser reactor at a first temperature and reactor effluent, comprising zeolite-comprising catalyst, is retrieved from the riser reactor at a second temperature. More preferably, the zeolite-comprising catalyst in the reactor effluent has a temperature equal to the second temperature. In the process according to the invention, it is preferred that sufficient C5 olefins are admitted to the reactor to maintain a temperature difference between the first and second temperature of in the range of from 0 to 40° C., preferably 0 to 30° C., more preferably 0 to 10° C.

As the reaction mixture passes through the reactor along the flow path, the temperature may initially rise as a result of the exothermic reaction taking place in the reaction mixture. Subsequently, the temperature of the reaction mixture decreases as the oxygenate becomes depleted and the endothermic reactions continue.

As a result the axial temperature profile, i.e., in the direction of the flow path, of the reaction mixture may go through a maximum downstream of the one or more upstream inlets. This maximum in axial temperature profile of the reaction mixture is referred to as the maximum temperature, which is the highest temperature to which the reaction mixture, and thus also the catalyst is exposed. Therefore, sufficient C5 olefins are admitted to the reactor to maintain a temperature difference between the maximum temperature and the lowest of the first or second temperature of in the range of from 0 to 40° C., preferably 0 to 30° C., more preferably 0 to 10° C.

The exact amount of C5 olefins that need to be admitted to the reactor may depend on many factors. One such factor is the extent of the conversion obtained, which may in turn depend on the catalyst activity and even on the type of zeolite-comprising catalyst used. The desired molar ratio of ethylene to propylene in the reactor effluent will also influence the amount of C5 that needs to be admitted. Typically, the choice of catalyst determines the obtained molar ratio of ethylene to propylene.

Another factor may be the choice of oxygenate and the presence of C4 olefins into the oxygenate feedstock. For instance the conversion of DME to olefins is less exothermic than the conversion of methanol to olefins and will therefore require less C5 olefin cracking to maintain the temperature of the reaction mixture below desirable levels. Also the presence of C4 olefins in the initial reaction mixture may influence the amount of C5 olefins required. The presence of C4 olefins will result in a lower temperature rise given that the C4 olefins following alkylation with the oxygenate are converted in a second endothermic step to ethylene and propylene.

Preferably, sufficient C5 olefins, and optionally C6 olefins, are admitted along the flow trajectory such that in the range of from 0.0005 to 2.0 mol, preferably of from 0.001 to 1 mol, of C5 olefins, and optionally C6 olefins, are admitted per mol of oxygenate provided to the process as part of the oxygenate feedstock. Where C4 olefins are provided to the process together or as part of the oxygenate feedstock less C5 olefins may be required compared to a process where the feed to the process comprises no C4 olefins. The same applies where the oxygenate feedstock comprises an ether, such as DME, as the reaction of the ether is less exothermic compared to for instance methanol.

In case of an oxygenate feedstock comprising methanol, preferably, at least 0.1 mol, more preferably 0.3 mol of C5 olefins, and optionally C6 olefins, per mol of methanol in the oxygenate feedstock are admitted along the flow trajectory. Determination of the exact amount of C5 olefin, and optionally C6 olefin that needs to be added is within the skills of the person skilled in the art.

During continuous operation of the process, variation in the operating temperature may occur, which are not related to the heat released by the exothermic conversion of the oxygenate. Such temperature variations may for instance be caused by unintentional upsets in the feed preheat or may be intentional for instance due to aging of the catalyst. In the latter case it may be desirable to increase the operating temperature to compensate for a reduced catalyst activity. It is an advantage of the current process that the amount of C5 olefins, and optionally C6 olefins, admitted to the process may remain essentially constant during such temperature variations.

Preferably, the amount of C5 olefins, and optionally C6 olefins, admitted to the process per mole of oxygenate is not changed more than 10 wt %, based on the original admitted amount of C5 olefins, and optionally C6 olefins, admitted to the process per mole of oxygenate, within a variation of the operation temperature in the range of from 0 to 100° C. More preferably, the amount of C5 olefins, and optionally C6 olefins, admitted to the process per mole of oxygenate is not changed more than 5 wt %, based on the original admitted amount of C5 olefins, and optionally C6 olefins, admitted to the process per mole of oxygenate, within a variation of the operation temperature in the range of from 0 to 100° C.

The reaction mixture passes through the riser reactor and exits the reactor as the reactor effluent. The reactor effluent comprises the zeolite-comprising catalyst and a gaseous product, comprising ethylene and propylene. The reactor effluent comprises advantageously at least 50 mol %, in particular at least 50 wt %, ethylene and propylene, based on total hydrocarbon content in the reactor effluent.

Typically, the gaseous product also comprises diluents provided to riser reactor together with or as part of the oxygenate feedstock or with the C5 olefins.

In addition to ethylene and/or propylene, the gaseous product may comprise higher olefins, i.e. C4+ olefins, and paraffins. The main by-products from the reaction are C4 and C5 olefins.

Preferably, the reactor effluent is subsequently provided to one or more gas/solid separators to retrieve zeolite-comprising catalyst from the reactor effluent.

The gas/solid separator may be any separator suitable for separating gases from solids. Preferably, the gas/solid separator comprises one or more centrifugal or cyclone, preferably cyclone, units, optionally combined with a stripper section.

In the gas/solid separator, a gaseous product is separated from the zeolite-comprising catalyst. The gaseous product is preferably further treated to retrieve several product fractions from the gaseous product. The product fractions will preferably comprise one or more fractions comprising ethylene and/or propylene. The separation of the gaseous product in the mentioned fractions may be done using any suitable work-up section. The design of the work-up section depends on the exact composition of the gaseous product, and may include several separation steps. The design of such a work-up section is well known in the art and does not require further explanation.

Preferably, the product fractions will also comprise one or more fractions comprising C4+ olefins and in particular C4 and C5 olefins.

Preferably, where the reactor effluent, and consequently the gaseous product, comprises C5 olefins, the process further comprises subjecting the reaction effluent, and ultimately the gaseous product, to one or more fractionation steps to retrieve at least a fraction comprising C5 olefins from the reaction effluent. Preferably, at least part of this fraction comprising C5 olefins is admitted to the riser reactor by providing at least part of the fraction comprising C5 olefins to the one or more locations along the length of the flow trajectory. This has the particular advantage that less C5 olefins need to be provided externally to the process. It is particularly preferred that all C5 olefins in the fraction comprising C5 olefins are admitted to the reactor at the one or more of locations along the length of the flow trajectory

Where another source of C5 olefins is available for admission to the riser reactor, there will be an excess of C5 olefins available and not all C5 olefins in the reactor effluent may be required to cool the reactor. Preferably, in that case a second part of the fraction comprising C5 olefins is provided to a second reactor, preferably also a riser reactor. In the second riser reactor, the fraction comprising C5 olefins is, preferably, contacted with a zeolite-comprising catalyst at a temperature in the range of from 500 to 700° C. From the second riser reactor a second reactor effluent may be retrieved comprising ethylene and/or propylene. The second reactor effluent comprises advantageously at least 50 mol %, in particular at least 50 wt %, ethylene and propylene, based on total hydrocarbon content in the second reactor effluent.

The conversion of the fraction comprising C5 olefins over a zeolite-comprising catalyst to at least ethylene and/or propylene is also referred to as an olefin cracking process (OCP). Such OCP processes are well known in the art.

Preferably, the same zeolite comprising catalyst is used to convert the oxygenates to olefins as well as the conversion of the fraction comprising C5 olefins in the second reactor. This has the particular advantage that the second reactor effluent may be provided to the same gas/solid separator as the reactor effluent obtained from the conversion of the oxygenates. In addition, other facilities such as catalyst regeneration facilities may be shared. Both mentioned synergies allow a reduction of the CAPEX.

As mentioned above, the reactor effluent may also comprise C4 olefins as part of the gaseous product. As described herein, C4 olefins may be reacted with oxygenates to form ethylene and propylene in an oxygenates to olefin process. As such, C4 olefins in the reactor effluent may be recycled back to the one or more upstream inlets of the riser reactor and be provided to the riser reactor as part of or together with the oxygenate feedstock to form part of the initial reaction mixture.

Therefore, preferably, where the reactor effluent, and consequently the gaseous product, further comprises C4 olefins, the process further comprises subjecting the reaction effluent, and ultimately the gaseous product, to one or more fractionation steps to retrieve at least a fraction comprising C4 olefins from the reaction effluent. Preferably, at least part of the fraction comprising C4 olefins is provided to the one or more upstream inlets of the riser reactor, together with or as part of the oxygenate feedstock to form part of the initial reaction mixture. This has the advantage that the C4 olefins may be converted, to provide further ethylene and propylene.

Although less desired, the gaseous product retrieved from the reactor effluent will typically comprise some aromatic compounds such as benzene, toluene and xylenes. Although it is not the primary aim of the process, xylenes can be seen as a valuable product. Xylenes are amongst others formed in the OTO process by the alkylation of benzene and, in particular, toluene with oxygenates such as methanol. Therefore, in a preferred embodiment, a separate fraction comprising aromatics, in particular benzene, toluene and xylenes is separated from the gaseous product and at least in part recycled to the riser reactor as part of or together with the oxygenate feedstock. Preferably, part or all of the xylenes in the fraction comprising aromatics are withdrawn from the process as a product prior to recycling the fraction comprising aromatic to the riser reactor.

The oxygenate feedstock provided to the process in the riser reactor comprises oxygenate. The oxygenate used in the oxygenate feedstock provided to the OTO process is preferably an oxygenate which comprises at least one oxygen-bonded alkyl group. The alkyl group preferably is a C1-C5 alkyl group, more preferably C1-C4 alkyl group, i.e. comprises 1 to 5, or 1 to 4 carbon atoms respectively; more preferably the alkyl group comprises 1 or 2 carbon atoms and most preferably one carbon atom. Examples of oxygenates that can be used in the oxygenate feedstock include alcohols and ethers. Examples of preferred oxygenates include alcohols, such as methanol, ethanol, propanol; and dialkyl ethers, such as dimethylether, diethyl ether, methylethyl ether. Preferably, the oxygenate is methanol or dimethylether, or a mixture thereof.

Preferably, the oxygenate feedstock comprises at least 50 wt. % of oxygenate, in particular methanol and/or dimethylether, based on total hydrocarbons, i.e. hydrocarbons including oxygenates, more preferably at least 70 wt. %.

Preferably, the oxygenate feedstock comprises oxygenate and olefins, more preferably oxygenate and olefins in an oxygenate:olefin molar ratio in the range of from 1000:1 to 1:1, preferably 100:1 to 1:1. More preferably, in a oxygenate:olefin molar ratio in the range of from 20:1 to 1:1, more preferably in the range of 18:1 to 1:1, still more preferably in the range of 15:1 to 1:1, even still more preferably in the range of 12:1 to 1:1. As mentioned above, it is preferred to convert a C4 olefin together with an oxygenate, to obtain a high yield of ethylene and propylene, therefore preferably at least one mole of oxygenate is provided for every mole of C4 olefin. The olefins comprised in the feedstock do not include the olefins, in particular C5 olefins, admitted to the reactor along the length of the flow trajectory downstream of the inlets for the oxygenate feedstock.

In the process the oxygenate feedstock is contacted with the zeolite-comprising catalyst. The oxygenate feedstock is contacted with the catalyst at a temperature in the range of from 500 to 700° C., more preferably of from 530 to 620° C., even more preferably of from 580 to 610° C.; and a pressure in the range of from 0.1 kPa (1 mbara) to 5 MPa (50 bara), preferably of from 100 kPa (1 bara) to 1.5 MPa (15 bara), more preferably of from 100 kPa (1 bara) to 300 kPa (3 bara). Reference herein to pressures is to absolute pressures.

The C5 olefins admitted to the reactor along the flow trajectory may be admitted as part of a C5 olefins-comprising feed or a C5-consisting feed. Where the C5 olefins are provided as part of a C5-comprising feed, preferably, such C5-comprising feed comprises in the range of from 50 to 100 wt % of C5 olefins, more preferably 80 to 100%, based on the C5-comprising feed. Other components in the C5-comprising feed may include diluents such as water or paraffins. Optionally, the C5-comprising feed further comprises C6 olefins. Still more preferably, the C5 olefins admitted to the reactor along the flow trajectory are admitted as part of a C5 and C6-consisting feed, even still more preferably a C5-consisting feed.

In the optional second reactor, the fraction comprising C5 olefins is contacted in an OCP process with a zeolite-comprising catalyst. The fraction comprising C5 olefins provided to the OCP process in the second reactor comprises C5 olefin. Preferably, the fraction comprising C5 olefins includes preferably C5+ olefins, most preferably includes C5 olefins.

Preferably, the fraction comprising C5 olefins comprises at least 50 wt. % of olefin, in particular C5 olefin, based on total hydrocarbons, more preferably at least 70 wt. %. In addition to the fraction comprising C5 olefins, other olefin comprising feedstocks may be provided to the second reactor.

The fraction comprising C5 olefins is contacted with the catalyst in the second reactor at a temperature of in the range of from 500 to 700° C., preferably of from 550 to 650° C., more preferably of from 550 to 620° C., even more preferably of from 580 to 610° C.; and a pressure in the range of from 0.1 kPa (1 mbara) to 5 MPa (50 bara), preferably of from 100 kPa (1 bara) to 1.5 MPa (15 bara), more preferably of from 100 kPa (1 bara) to 300 kPa (3 bara). Reference herein to pressures is to absolute pressures. As the cracking of olefins, in particular C5 olefins is an endothermic process it is generally not required to provide measures to reduce the temperature in the second reactor.

As mentioned above the process is operated using riser reactors. The primary operators for controlling the reaction inside the reactor, and in particular a riser reactor, are the gas residence time, the cat/oil ratio and the feed and catalyst inlet temperature. The gas residence time and the cat/oil ratio may be correlated to the earlier mentioned WHSV.

The gas residence time herein refers to the average time it takes for gas at the reactor, inlet to reach the reactor outlet. The gas residence time is also referred to as τ. The dimensionless cat/oil ratio herein refers to the mass flow rate of catalyst (kg/h) divided by the mass flow rate of the feed (kg/h), wherein the flow rate of the feed is calculated on a CH₂ basis.

In addition to the oxygenates and olefins, also an amount of diluent is provided to the riser reactor together with or as part of the oxygenate feedstock.

During the conversion of the oxygenates in the riser reactor, steam is produced as a by-product, which serves as an in-situ produced diluent. Typically, additional steam is added as diluent. The amount of additional diluent that needs to be added depends on the in-situ water make, which in turn depends on the composition of the oxygenate feedstock. Where the diluent provided to the riser reactor is water or steam, the molar ratio of oxygenate to diluent is between 10:1 and 1:20. Other suitable diluents include inert gases such as nitrogen or methane, but may also include C2-C3 paraffins.

A diluent may also be provided to the second reactor together with the olefins. Preferably, the diluent provided to the second reactor is water or steam. Other suitable diluents include inert gases such as nitrogen or methane, but may also include C2-C3 paraffins. Preferably, the diluents provided to the first and second reactor are the same, more preferably water or steam.

The zeolite-comprising catalyst is a zeolite-comprising catalyst suitable for converting the oxygenates and olefins and preferably includes zeolite-comprising catalyst compositions. Such zeolite-comprising catalyst compositions typically also include binder materials, matrix material and optionally fillers. Suitable matrix materials include clays, such as kaolin. Suitable binder materials include silica, alumina, silica-alumina, titania and zirconia, wherein silica is preferred due to its low acidity.

Zeolites preferably have a molecular framework of one, preferably two or more corner-sharing [TO₄] tetrahedral units, more preferably, two or more [SiO₄], [AlO₄] tetrahedral units.

The zeolite-comprising catalysts include those catalyst containing a zeolite of the ZSM group, in particular of the MFI type, such as ZSM-5, the MTT type, such as ZSM-23, the TON type, such as ZSM-22, the MEL type, such as ZSM-11, the FER type. Other suitable zeolites are for example zeolites of the STF-type, such as SSZ-35, the SFF type, such as SSZ-44 and the EU-2 type, such as ZSM-48.

Under the appropriate reaction conditions, these catalyst may induce the cracking of olefins as well as the conversion of oxygenates alone or together with C4 olefins to ethylene and propylene. These zeolite-comprising catalysts, in particular the ZSM zeolite-comprising catalyst have an advantage over for instance non-zeolite-comprising catalyst such as silicoaluminophosphates like SAPO-34. Although both types of catalyst are suitable to convert oxygenates to olefins, non-zeolite-comprising catalyst are less suitable for cracking olefins or converting oxygenates together with olefins such as C4 olefins. The advantage of using zeolites compared to e.g. silicoaluminophosphates becomes even more pronounced when the olefins include iso-olefins such as isobutene.

Preferred catalysts comprise a more-dimensional zeolite, in particular of the MFI type, more in particular ZSM-5, or of the MEL type, such as zeolite ZSM-11. The zeolite having more-dimensional channels has intersecting channels in at least two directions. So, for example, the channel structure is formed of substantially parallel channels in a first direction, and substantially parallel channels in a second direction, wherein channels in the first and second directions intersect. Intersections with a further channel type are also possible. Preferably the channels in at least one of the directions are 10-membered ring channels. A preferred MFI-type zeolite has a Silica-to-Alumina ratio SAR of at least 60, preferably at least 80.

The zeolite-comprising catalyst may comprise more than one zeolite. In that case it is preferred that the catalyst comprises at least a more-dimensional zeolite, in particular of the MFI type, more in particular ZSM-5, or of the MEL type, such as zeolite ZSM-11, and a one-dimensional zeolite having 10-membered ring channels, such as of the MTT and/or TON type.

The zeolite-comprising catalyst may comprise phosphorus as such or in a compound, i.e. phosphorus other than any phosphorus included in the framework of the zeolite. It is preferred that a catalyst comprising a MEL or MFI-type zeolite additionally comprises phosphorus. The phosphorus may be introduced by pre-treating the MEL or MFI-type zeolites prior to formulating the catalyst and/or by post-treating the formulated catalyst comprising the MEL or MFI-type zeolites. Preferably, the catalyst comprising MEL or MFI-type zeolites comprises phosphorus as such or in a compound in an elemental amount of from 0.05 to 10 wt % based on the weight of the formulated catalyst. A particularly preferred catalyst comprises phosphor and MEL or MFI-type zeolites having SAR of in the range of from 60 to 150, more preferably of from 80 to 100. An even more particularly preferred catalyst comprises phosphor and ZSM-5 having SAR of in the range of from 60 to 150, more preferably of from 80 to 100.

It is preferred that zeolites in the hydrogen form are used in the zeolite-comprising catalyst, e.g., HZSM-5, HZSM-11, and HZSM-22, HZSM-23. Preferably at least 50 wt %, more preferably at least 90 wt %, still more preferably at least 95 wt % and most preferably 100 wt % of the total amount of zeolite used is in the hydrogen form. It is well known in the art how to produce such zeolites in the hydrogen form.

Preferably, the zeolite-comprising catalyst containing phosphorus has been prepared by a process which includes at least the following steps:

i) preparing an aqueous slurry comprising a zeolite, clay material and binder;
ii) spraydrying the aqueous slurry to obtain zeolite-comprising catalyst particles;
iii) treating the spraydried zeolite-comprising catalyst particles with phosphoric acid to introduce phosphorus compounds on the spraydried and zeolite-comprising catalyst particles; and
iv) calcining the spraydried zeolite- and phosphorus-comprising catalyst particles.

Preferably, the residence time of the reaction mixture in the riser reactor, also referred to as τ, is in the range of from 1 to 10 seconds, more preferably of from 3 to 6 seconds, even more preferably of from 3.5 to 4.5 seconds.

Preferably, the cat/oil ratio i.e. on a CH₂ basis for hydrocarbons including oxygenates, in the riser reactor is in the range of from 1 to 100, more preferably of from of from 1 to 50, even more preferably 5 to 25.

It is preferable to control the severity of the process in the riser reactor. When the process is operated at a too high severity, side reactions increase as well as by-product formation at the cost of ethylene and propylene selectivity. In case, the severity is too low, the process is operated inefficient and suboptimal conversions are obtained. The severity of the process is influenced by several reaction and operation conditions, however, a suitable measure for the severity of the process in the riser reactor is the C5 olefin content in the reactor effluent. A higher C5 olefin content indicates lower severity and vice versa. Preferably, the reaction conditions in the riser reactor are chosen such that the reactor effluent comprises in the range of from 2.5 to 40 wt % of C5 olefins, based on the hydrocarbons in the reactor effluent, preferably 4 to 15 wt % of C5 olefins. The C5 content in the reactor effluent is conveniently analyzed using any suitable means of analyzing the hydrocarbon content in a process stream. Particularly suitable means of analyzing the C5 content in the reactor effluent include gas chromatography and near infrared spectroscopy.

Preferably, the reaction conditions in the riser reactor are chosen such that the oxygenate conversion is in the range of from 90 to 100%, based on the oxygenates provided to the riser reactor, preferably 95 to 100%.

In addition to the deactivation of the catalyst due to the exposure to high temperature, the catalyst is subject to another, though reversible, deactivation process caused by the deposition of coke on the catalyst in the course of the process. The catalyst may be regenerated by an oxidative regeneration process, whereby at least part of the coke deposits on the catalyst are oxidized. Regenerated catalyst can be recycled to the process of the invention.

The catalyst particles used in the process of the present invention can have any shape known to the skilled person to be suitable for this purpose, for example, it can be present in the form of spray dried catalyst particles which can be spheres. Typically and preferably, Geldart A—class particles are used, see D. Kunii and O. Levenspiel, Fluidization Engineering, 2^{nd} Ed, Butterworth-Heineman, Boston, London, Singapore, Sydney, Toronto, Wellington, 1991, p 77 for Geldart classification of particles. Spray-dried particles allowing use in a fluidized bed or riser reactor system are preferred. Spherical particles are normally obtained by spray drying. Preferably the average particle size is in the range of 1-200 μm, preferably 50-100 μm.

The invention also provides a reaction system suitable for preparing ethylene and propylene. The system according to the invention is herein below explained in more detail with reference to the non-limiting Figures.

The person skilled in the art will readily understand that many modifications may be made without departing from the scope of the present invention. Further, the person skilled in the art will readily understand that, while the present invention in some instances may have been illustrated making reference to a specific combination of features and measures, many of those features and measures are functionally independent from others features and measures given in the respective embodiment(s) such that they can be equally or similarly applied independently in other embodiments.

In FIG. 1, a non-limiting schematic representation is provided of a process according to the invention. In the process, a riser reactor 10 is provided, which comprises a riser wall 15. Riser wall 15 defines a flow trajectory 20 through the riser reactor 10. In riser reactor 10, a first riser reactor stage 22a is defined and downstream of first riser reactor stage 22a, one or more subsequent riser reactor stages 22b are defined. An oxygenate feedstock 25 is provided to first riser reactor stage 22a of riser reactor 10. In addition, zeolite-comprising catalyst 30 is also provided to first riser reactor stage 22a or riser reactor 10. The oxygenate feedstock and zeolite-comprising catalyst form a reaction mixture that passes along flow trajectory 20 toward and through the one or more subsequent riser reactor stages 22b, at a temperature in the range of from 500 to 700° C. Reactor effluent 35 is retrieved from riser reactor 10 and is passed to gas/solid separator 40.

In gas/solid separator 40, the zeolite-comprising catalyst is separated from a gaseous product. The zeolite-comprising catalyst 45 is retrieved from the gas/solid separator 40 and may be provided to one or more of a catalyst regeneration facility (not shown), another reactor (not shown) or may be recycled to riser reactor 10 as, or as part of zeolite-comprising catalyst 30. The gaseous product 50 retrieved from gas/solid separator 40 may be provided to a separation section 60. In separation section 60 the gaseous product is treated to remove steam and water and to separate the remainder into the desired product fractions. Such treatment may include for instance a water quench to remove steam and one or more compression steps to compress the gaseous product. Typically, at least one or more fractions comprising ethylene and propylene 65 are retrieved from separation section 60. However, preferably, also a fraction comprising C5 olefins (62 & 70) is retrieved. This fraction comprising C5 olefins 62 may be provided back to riser reactor 10 and admitted to riser reactor 10 at locations 75a, 75b, 75c, and 75d, and optionally further locations (not shown), along flow trajectory 20 and in the one or more subsequent riser reactor stages 22b. Preferably, a part of the fraction comprising C5 olefins 70 is removed from the process as purge stream 72, to prevent the build-up of inert C5 paraffins in the process. Optionally, additional external C5 olefins 77 are provided to locations 75a, 75b, 75c, and 75d, and optionally further locations (not shown).

If desired, part 85 of fraction comprising C5 olefins 70 is provided to second riser reactor 80. In second reactor 80, part 85 of the fraction comprising C5 olefins is contacted with zeolite-comprising catalyst 90 at a temperature in the range of from 500 to 700° C.

In the herein described embodiment zeolite-comprising catalyst 30 and zeolite-comprising catalyst 90 are the same. Therefore, a second reactor effluent 95 may be retrieved from second riser reactor 80 and provided to gas/solid separator 40 to be separated together with reactor effluent 35 from riser reactor 10.

In addition, preferably, also a fraction comprising C4 olefins 100 is retrieved from separation section 60 and provided back to riser reactor 10 as part of the oxygenate feed 25. Preferably, a part of fraction comprising C4 olefins 100 is removed from the process as purge stream 105, to prevent the build-up of inert C4 paraffins in the process.

FIG. 2 shows an embodiment of a reaction system according to the invention, suitable for preparing ethylene and propylene. Riser reactor 10 comprises an inlet for oxygenate feedstock 225 and an inlet for zeolite catalyst 230. Riser reactor 10 further comprises an outlet for reactor effluent 235.

In riser reactor 10, a first riser reactor stage 22a is defined and downstream of first riser reactor stage 22a, one or more subsequent riser reactor stages 22b are defined. Reactor wall 15 defines a flow trajectory 20 from the inlets 225 to the outlet 235, which passes through the one or more subsequent riser reactor stages 22b.

Riser reactor 10 further comprises at least one inlet array 275 for providing C5 olefins into the reactor, integrated with the reactor wall 15.

In FIG. 2a, a more detailed drawing is provided of a preferred embodiment of an inlet array 275 as may be used in the reactor system according to the invention. The inlet array 275 comprises one or more inlets 300 for C5 olefins. Preferably, the inlet array 275 comprises a plurality of inlets for C5 olefins 300 distributed along the periphery 310 of the reactor wall 15. Reference herein to inlet array integrated with the reactor wall is to an inlet array that is an integral part of the reactor wall or to an inlet array that is arrange on the inner side of the reactor wall along the periphery of the reactor wall.

FIG. 2a further shows an obstructing member 320 extending from the reactor wall 15 into the flow trajectory 20.

The obstructing member 320 is preferably a ring shaped device, which when placed inside a riser reactor results in a localised decreased inner diameter of the reactor cross-section. The decreased inner diameter being determined by a circular or oval opening 330 in the obstructing member, wherein the central axis of the opening is aligned along the central axis 340 of the riser reactor. Preferably, the obstructing member is a metal or ceramic ring that is attached to the inner side of the reactor wall. Preferably, the obstructing member causes the inner diameter to be locally decreased by in the range of from 1 to 25%, preferably of from 2 to 10%, based on the inner diameter of the riser reactor.

Preferably, the obstruction member is arranged upstream of the one or more inlets for C5 olefins with respect to the at least one inlet for oxygenate feed or with respect to the flow trajectory 20.

The invention further provides the use of the reaction system according to the invention in a process according

### EXAMPLES

The invention is illustrated by the following non-limiting calculated examples.

Tables 1 and 2 show the required amount (mol) of C5 olefin per mol of methanol converted that needs to be admitted to the process in order to operate the process isothermally, i.e. where zeolite-comprising catalyst is provided to the riser reactor at a first temperature and reactor effluent, comprising zeolite-comprising catalyst, is retrieved from the riser reactor at a second temperature and sufficient C5 olefins are admitted to the reactor to maintain a temperature difference between the first and second temperature that is zero. The calculations are based on the heat of formation (ΔH_{f}(T) (kJ/mol)) and ratio of ethylene to propylene in the reactor effluent. It is assumed that the only reactants are methanol and 1-pentene, which are converted as following:

MeOH->H₂O+nC₂H₄+mC₃H₆ (1a)

C5H_{10->}C₂H₄+C₃H₆ (1b)

3m+2n=1 (1c)

(0≦m≦⅓) (1d)

The only products formed are ethylene, propylene and water. The C5 olefins are admitted having a temperature equal to the temperature of the reaction mixture.

In Table 1, calculated values for the heat of formation (ΔH_{f}(T) (kJ/mol)) of the several reactants and products are shown at different temperatures. In Table 2, the amount (in mol) of C5 olefins that needs to be added to attain a temperature increase of the reactor of zero is calculated for two different ethylene to propylene molar ratio's in the reactor effluent.

It will be clear from Tables 1 and 2, that the heat of reaction released by the exothermic conversion of methanol to ethylene and propylene can be absorbed by cracking C5 olefins in the same process to prevent or at least reduce the temperature increase during the process that would have been observed in the absence of the C5 olefins cracking.

Furthermore, it will be clear that the moles of 1-pentene required to be admitted to the process is essentially independent of the operating temperature.

TABLE 1

| ΔH_{f} (T) | T [° C.] | | | | |
|---|---|---|---|---|---|
| [kJ/mol] | 500 | 530 | 600 | 620 | 700 |
| MeOH | −214.28 | −214.79 | −215.84 | −216.10 | −216.98 |
| H₂O | −246.32 | −246.56 | −247.10 | −247.25 | −247.83 |
| C2= | 41.22 | 40.77 | 39.84 | 39.61 | 38.80 |
| C3= | 3.72 | 3.09 | 1.82 | 1.51 | 0.49 |
| 1-C5= | −45.80 | −46.62 | −48.19 | −48.55 | −49.60 |

TABLE 2

| | T [° C.] | | | | |
|---|---|---|---|---|---|
| C₂⁼/C₃⁼ | 1-C₅⁼/MeOH [mol/mol] | | | | |
| [mol/mol]^{#} | 500 | 530 | 600 | 620 | 700 |
| 1:1 | 0.25 | 0.25 | 0.25 | 0.26 | 0.26 |
| 3:7 | 0.29 | 0.29 | 0.29 | 0.29 | 0.29 |

^{#}In the reactor effluent

What is claimed is:

1. A process for preparing ethylene and/or propylene, said process comprising contacting an oxygenate feedstock with a zeolite-comprising catalyst at a temperature in the range of from 500 to 700° C. to obtain a reactor effluent comprising ethylene and/or propylene wherein the oxygenate feedstock is contacted with the catalyst in a riser reactor having a reactor wall defining a flow trajectory towards a downstream outlet for reactor effluent, and wherein at least oxygenate feedstock and catalyst are provided at one or more upstream inlets of the riser reactor and wherein C5 olefins are admitted to the riser reactor at one or more of locations along the length of the flow trajectory.

2. A process according to claim 1, wherein the C5 olefins are admitted to the riser reactor at two or more of locations along the flow trajectory, preferably the C5 olefins are admitted to the riser reactor in the range of from 2 to 15 locations along the length of the flow trajectory.

3. A process according to claim 1, wherein one or more obstructing members are provided extending from the reactor wall into the flow trajectory and wherein the C5 olefins are admitted downstream of the obstructing members, with respect to the flow trajectory.

4. A process according to claim 1, wherein at each location where C5 olefins are admitted to the reactor, the C5 olefins are admitted through a plurality of inlets distributed along the periphery of the reactor wall.

5. A process according to claim 1, wherein the zeolite-comprising catalyst is provided to the riser reactor at a first temperature and reactor effluent, comprising zeolite-comprising catalyst, is retrieved from the riser reactor at a second temperature and sufficient C5 olefins are admitted to the reactor to maintain a temperature difference between the first and second temperature of in the range of from 0 to 40° C.

6. A process according to claim 1, wherein the reactor effluent comprises C5 olefins and the process further comprises subjecting the reaction effluent to one or more fractionation steps to retrieve at least a fraction comprising C5 olefins from the reaction effluent and admitting at least part of the fraction comprising C5 olefins to the riser reactor by providing at least part of the fraction comprising C5 olefins to the one or more of locations along the length of the flow trajectory.

7. A process according to claim 6, comprising providing a second part of the fraction comprising C5 olefins to a second reactor, contacting the fraction comprising C5 olefins with a zeolite-comprising catalyst at a temperature in the range of from 500 to 700° C. and retrieving from the second reactor a second reactor effluent stream comprising ethylene and/or propylene.

8. A process according to claim 1, wherein the reactor effluent comprises C4 olefins and the process further comprises subjecting the reaction effluent to one or more fractionation steps to retrieve at least a fraction comprising C4 olefins from the reaction effluent and providing at least part of the fraction comprising C4 olefins to the one or more upstream inlets of the riser reactor, together with or as part of the oxygenate feedstock.

9. A process according to claim 1, wherein the zeolite-comprising catalyst comprises ZSM-5.

10. A process according to claim 1, wherein the oxygenate feedstock comprises methanol and/or dimethylether.

11. A process according to claim 1, wherein the C5 olefins are admitted as part of a C5 olefins-comprising feed, which C5 olefins-comprising feed further comprises C6 olefins.

12. A reaction system suitable for preparing ethylene and propylene, comprising a riser reactor comprising:
a) an inlet for oxygenate feedstock;
b) an inlet for zeolite catalyst;
c) an outlet for reactor effluent;
d) a reactor wall defining a flow trajectory from the inlets for oxygenate feedstock and zeolite catalyst to the outlet for reactor effluent; and
e) at least one inlet array for providing C5 olefins into the reactor, integrated with the reactor wall.

13. A reaction system according to claim 12, wherein the inlet array comprises one or more inlets for C5 olefins and an obstructing member extending from the reactor wall into the flow trajectory, wherein the obstruction member is arranged upstream of the one or more inlet for C5 olefins with respect to at least one inlet for oxygenate feedstock.

14. A reaction system according to claim 12, wherein the inlet array comprises a plurality of inlets for C5 olefins distributed along the periphery of the reactor wall.
